(19) United States
(12) Patent Application Publication
(10) Pub. No.: US 2024/0207141 A1
(43) Pub. Date: Jun. 27, 2024

(54) PEPTIDE HAVING REPARATIVE DENTIN-REGENERATING ACTIVITY AND USE THEREOF

(71) Applicant: SEOUL NATIONAL UNIVERSITY R&DB FOUNDATION, Seoul (KR)

(72) Inventors: Byung-Moo MIN, Seoul (KR); Sun-Young KIM, Seoul (KR); Choyeon PARK, Osan-si, Gyeonggi-do (KR)

(73) Assignee: SEOUL NATIONAL UNIVERSITY R&DB FOUNDATION, Seoul (KR)

(21) Appl. No.: 18/557,532

(22) PCT Filed: Mar. 6, 2023
(86) PCT No.: PCT/KR2023/003027

§ 371 Date: Oct. 26, 2023

(30) Foreign Application Priority Data
Mar. 11, 2022 (KR) 10-2022-0031002

(51) Int. Cl. A61K 6/69; A61K 38/10; A61K 38/16; A61P 1/02; C12N 5/077; C12N 15/63

(52) U.S. Cl. CPC A61K 6/69; A61K 38/10; A61K 38/16; A61P 1/02; C12N 5/0654; C12N 15/63; C12N 2501/998

(57) ABSTRACT

The present invention relates to a peptide having dentin-regenerating activity and the use thereof. The peptide of the present invention promotes the behavior of dental pulp cells, including attachment, spreading and migration of dental pulp cells, and promotes the differentiation of dental pulp cells into odontoblasts. The peptide of the present invention may be used as an active ingredient of a dental material related to tooth restoration and dentin regeneration, for example, a root canal filling material or a pulp-capping agent, and may also be used for the treatment and prevention of dental pulp disease.

### TECHNICAL FIELD

### Cross-Reference to Related Applications

This application claims the benefit of priority based on Korean Patent Application No. 10-2022-0031002 filed on Mar. 11, 2022, and the entire contents of which are incorporated as part of the present specification.

### Technical Field

The present invention relates to dentin regeneration of vitronectin-derived functional peptides, prevention or treatment of dental pulp disease, and the use of pulp-capping agents and root canal filling materials.

### BACKGROUND ART

A tooth is an organ having enamel in the outermost layer, a hard tissue called dentin in its inner layer, odontoblasts producing dentin inside the dentin, and dental pulp cells in the core. Dental pulp is a soft connective tissue filling the pulp chamber inside the tooth and is richly distributed with nerves and blood vessels, and refers to a place that reaches the surface layer of dentin. A pulp-dentin complex is a very dynamic functioning structure and is a tissue that plays a very important role in protecting teeth, such as immunologically defending against noxious stimuli and responding to trauma or infection by healing and regenerating. Dentin is classified into primary dentin, secondary dentin and tertiary dentin according to the order of development. Tertiary dentin is additional dentin formed on the dental pulp side in response to noxious stimuli such as dental caries, attrition, abrasion, and restorative dental procedures applied to the teeth. This dentin is formed only in areas where the existing dentin has been subjected to noxious stimuli. There are two types of tertiary dentin: reactionary dentin and reparative dentin. Reactionary dentin is dentin formed by existing odontoblasts when noxious stimuli are weak, and reparative dentin is dentin formed by the death of the existing odontoblasts that produced primary dentin and secondary dentin due to severe noxious stimuli and the new differentiation of odontoblast-like cells. Dental pulp disease caused by bacterial infection of the dental pulp due to damage to dentin may extend to periapical disease and periodontal disease.

The tooth is formed by the induction of prenatal developmental processes, is a functional unit constructed by several cell types, and is like an organ or viscera. Thus, a tooth is not generated by a stem cell system that develops into various cell types from stem cells such as hematopoietic stem cells in an adult or mesenchymal stem cells. As such, the tooth has the characteristic of regenerating spontaneously in an adult, but is a tissue that is continuously exposed to the outside and used in daily life such as human food intake and language life, and is an organ that may be easily lost due to dental caries, periodontal disease, external shock, or the like. Accordingly, due to the characteristic of tissue that does not regenerate spontaneously, recently, it is often supplemented with methods such as dentures or implants. However, since the presence or absence of teeth greatly affects the appearance or taste of food, interest in the development of dental tissue regeneration technology is increasing for health maintenance and high quality life.

Gutta percha, which is a conventional root canal filling material used for dental caries treatment, has the advantages of good plasticity and operability, low cytotoxicity, radiopacity, and easy removal by heat and solubilizers, but has a risk of micro-leakage because it does not adhere to dentin. In addition, micro-leakage occurs because it contracts as it cools when heated, it is very difficult to use in wide root canals or unfinished roots because it has only the ability to physically close the root canal and does not have the ability to induce dentin, and failure of dental caries treatment is inevitable if necrosis occurs before reparative dentin is formed. In addition, when it is overfilled, it is difficult to remove and rather may cause periapical disease, so improvement has been required.

Efforts have been made to develop active substances such as peptides for dental pulp tissue regeneration, for example, Korean Patent No. 10-1943978 proposes a peptide having an activity to promote the regeneration of hard tissue and dental pulp tissue, and Japanese Patent No. 5519121 proposes a synthetic peptide containing an RGD sequence of dentin phosphophoryn having a hard tissue-inducing activity, but development of biocompatible active substances for dentin regeneration has not yet yielded satisfactory results.

Under this background, as a result of research efforts to develop a material for dentin regeneration, the present inventors have completed the present invention by experimentally proving that vitronectin-derived physiologically active peptides are active in promoting the differentiation of dental pulp cells into odontoblasts, cell behavior and dentin mineralization in a pulp exposure animal model.

### PRIOR ART DOCUMENTS

### Patent Documents

Korean Patent No. 10-1943978
Japanese Patent No. 5519121

### DISCLOSURE

### Technical Problem

It is an object of the present invention to provide a composition for regenerating dentin, comprising a vitronectin-derived physiologically active peptide as an active ingredient.

In addition, it is another object of the present invention to provide a pharmaceutical composition for preventing or treating dental pulp disease, comprising the peptide as an active ingredient.

In addition, it is still another object of the present invention to provide a composition for a root canal filling material, comprising the peptide as an active ingredient.

In addition, it is yet another object of the present invention to provide a composition for a pulp-capping agent, comprising the peptide as an active ingredient.

### Technical Solution

An aspect of the present invention provides a composition for regenerating dentin, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

Another aspect of the present invention provides a composition for preventing or treating dental pulp disease, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

Another aspect of the present invention provides a composition for a root canal filling material, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

Another aspect of the present invention provides a composition for a pulp-capping agent, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

Hereinafter, the present invention will be described in detail.

### Composition for Regenerating Dentin

An aspect of the present invention provides a composition for regenerating dentin, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

In the present invention, the term “peptide” refers to a polymer compound in which two or more amino acids are linked by a peptide bond.

In the present invention, the peptide used for dentin regeneration is a vitronectin-derived physiologically active peptide, which is a peptide consisting of 12 to 173 contiguous amino acid sequences comprising the amino acid sequence of SEQ ID NO: 1 (RVYFFKGKQYWE) within the amino acid sequence of SEQ ID NO: 2.

In other words, the peptide of the present invention is a peptide comprising the amino acid sequence of SEQ ID NO: 1 (RVYFFKGKQYWE), and may consist of 12 to 173 contiguous amino acid sequences within the amino acid sequence of SEQ ID NO: 2.

The peptide of the present invention is a peptide consisting of 12 to 173, 12 to 80, 12 to 40, 12 to 30, 12 to 25, 12 to 20, 12 to 14, or 12 to 13 contiguous amino acids comprising the amino acid sequence of SEQ ID NO: 1 within the amino acid sequence of SEQ ID NO: 2, and may be a peptide exhibiting dentin regeneration activity.

The peptide of the present invention is included in the scope of the peptide of the present invention as long as it exhibits dentine regeneration activity even when other amino acids are added to the amino acid sequence constituting the peptide of the present invention, amino acids are deleted therefrom, or other peptides are fused thereto.

The peptide consisting of the amino acid sequence of SEQ ID NO: 2 is a vitronectin-derived active peptide, which is a peptide from which a biologically active peptide motif consisting of the amino acid sequence of SEQ ID NO: 1 of the present invention is derived.

Since the peptide consisting of 12 to 173 contiguous amino acid sequences comprising the amino acid of SEQ ID NO: 1 within the amino acid sequence of SEQ ID NO: 2 comprises the biologically active peptide motif, it equally retains the activity of a peptide consisting of the amino acid sequence of SEQ ID NO: 1 (RVYFFKGKQYWE) of the present invention or a peptide comprising this amino acid sequence.

In the present invention, the peptide used for dentin regeneration is a vitronectin-derived physiologically active peptide, which is a peptide comprising the amino acid sequence of SEQ ID NO: 1 “RVYFFKGKQYWE”.

The peptide may include a variant peptide having a different sequence by deletion, insertion or substitution of amino acid residues, or a combination thereof, within the scope that does not affect the activity of the peptide, or may be in the form of a protein fragment having the same function.

Amino acid modifications at the protein and peptide level, which comprise the amino acid sequence of SEQ ID NO: 1 but do not entirely altering its activity, are known in the art, and in some cases, it may be modified by phosphorylation, sulfation, acrylation, glycosylation, methylation, farnesylation, and the like. Accordingly, the peptide comprises a peptide having not only the amino acid sequence of SEQ ID NO: 1 but also an amino acid sequence substantially identical thereto, or a variant thereof. The peptide having the substantially identical amino acid sequence may be, but is not limited to, a peptide comprising an amino acid sequence having 90% or more, 91% or more, 92% or more, 93% or more, 94% or more, 95% or more, 96% or more, 97% or more, 98% or more, 99% or more, or 99.5% or more homology to the amino acid sequence of SEQ ID NO: 1, and is included in the scope of the present invention as long as it includes a sequence having 90% or more homology to the amino acid sequence of SEQ ID NO: 1 and has the same activity.

The peptide of the present invention may consist of 20 or less amino acids showing dentin regeneration activity while including the amino acid sequence of SEQ ID NO: 1. Specifically, the peptide may consist of 20 or less, 18 or less, 15 or less, or 12 amino acids.

In one embodiment, the peptide may be a peptide having the amino acid sequence of SEQ ID NO: 1. In the present specification, the peptide having the amino acid sequence of SEQ ID NO: 1 is also referred to as “VnP-16 peptide” and described.

The peptide of the present invention may be obtained by various methods well known in the art. As an example, it may be prepared using polynucleotide recombination and protein expression systems, or may be prepared by an in vitro synthesis method through chemical synthesis such as peptide synthesis, a cell-free protein synthesis method, and the like, but is not limited by these preparation methods.

In addition, in order to obtain better chemical stability, enhanced pharmacological properties (half-life, uptake, titer, efficacy, and the like), altered specificity (for example, a broad spectrum of biological activity), and reduced antigenicity, a protecting group may be bound to the N-terminus or C-terminus of the peptide of the present invention. For example, the protecting group may be an acetyl group, a fluorenylmethoxycarbonyl group, a formyl group, a palmitoyl group, a myristyl group, a stearyl group, or polyethylene glycol (PEG), but any component capable of enhancing the modification of the peptide, in particular, the stability of the peptide may be included without limitation. The “stability” refers to not only in vivo stability that protects the peptide of the present invention from attack by in vivo protein cleaving enzymes, but also storage stability (for example, room temperature storage stability).

Since the peptide of the present invention has an activity to promote the regeneration of dentin by odontoblasts or dental pulp cells, it may be used for dentin regeneration or dentin regeneration promotion.

The term “dentin” of the present invention is a white hard tissue constituting most of a tooth, which is a mineralized tissue originating from the ectomesenchyme and containing collagen. Dentin is produced by odontoblasts originating from ectomesenchymal cells of dental papilla. Dentin is covered with enamel in the dental crown area and cementum in the tooth root area, so it is not exposed on the surface of the tooth, but when enamel wears away with aging, dentin is exposed at the tip or occlusal surface of the dental crown. Dentin differs from normal bone in that the body of the cell that produces dentin is present in the dental pulp, and only its process protrudes into the dentin. Since the odontoblasts that form dentin are present in the dental pulp, the dental pulp and dentin are considered to be a dentin-pulp complex, which is one functional unit.

In the present invention, the term “dental pulp” is a part consisting of soft connective tissue and odontoblasts filling the pulp chamber inside the tooth, is richly distributed with nerves and blood vessels, and is responsible for forming dentin.

In the present invention, the dentin may be reparative dentin or reactionary dentin. The peptide of the present invention promotes the regeneration or formation of dentin, specifically reparative dentin or reactive dentin. Reparative dentin and reactionary dentin are a type of tertiary dentin, and tertiary dentin is additional dentin formed on the dental pulp side in response to noxious stimuli such as dental caries, attrition, abrasion, and restorative dental procedures applied to the teeth. The restorative dentin is formed only in areas where the existing dentin has been subjected to noxious stimuli, and is dentin formed by the death of the existing odontoblasts that produced primary dentin and secondary dentin and the new differentiation of odontoblast-like cells from the dental pulp cells. The reactionary dentin is dentin formed by existing postmitotic odontoblasts that survive noxious stimuli when the noxious stimuli are weak. In the present invention, the dentin may be dental pulp-exposed dentin. Specifically, the dentin may be one in which the tooth is damaged and the dental pulp inside the tooth is exposed to the outside. In the examples of the present specification, it was confirmed that the peptide of the present invention promoted the formation of homogeneous and thick hard tissue in a rat pulp exposure model. The composition for regenerating dentin according to the present invention may be used for pulp-capping. In the present invention, the term “odontoblast” is present on the surface of the dental papilla in contact with the inner enamel cells of dentin, and the odontoblasts produce predentin, upon which the calcareous material is deposited to form dentin. In the present invention, the odontoblasts may include odontoblast-like cells as well as postmitotic odontoblasts. The odontoblast-like cells originally refer to cells induced to differentiate into odontoblasts from original stem cells, progenitor cells, dental pulp cells, or the like, and cells having characteristics similar to those of odontoblasts.

The peptide of the present invention has an activity that promotes dental pulp cell behavior. Specifically, the activity that promotes pulp cell behavior may be an activity that promotes the attachment, spreading or migration of dental pulp cells.

The peptide of the present invention exerts an activity to promote the regeneration of dentin through an activity that promotes the attachment, spreading or migration of dental pulp cells.

The term “attachment” of the present invention refers to the attachment of cells, and includes not only the attachment of cells to each other but also the attachment of cells to a matrix. When the attachment ability of cells is improved, cells are easily attached to the corresponding site.

The term “spreading” of the present invention refers to a process in which animal cells adhere to a matrix and then spread the cytoplasm thinly on the surface of the matrix, or a state in which cells have completed cell attachment to the matrix. Normal cells are dependent on the base, and thus cannot proliferate unless they are stretched by adhering to the matrix. Cell spreading occurs when integrin, which is a cell adhesion receptor on the cell surface, recognizes and adheres to cell adhesion proteins and the like on the matrix, and the adhesion signal is transmitted into the cell to orient the cytoskeleton and spread the cytoplasm thinly on the surface of the matrix. Cell spreading is distinct from cell attachment, and is related to cell proliferation and migration.

The term “migration” of the present invention means that cells migrate in response to external signals. When cell migration is improved, it may be helpful in inhibiting cell damage, and the ability to heal wounds may be excellent when there are noxious stimuli or wounds.

As demonstrated in one example of the present invention, the peptide of the present invention strongly promoted the attachment, spreading and migration of human dental pulp cells.

The peptide of the present invention has an activity that promotes the differentiation of dental pulp cells into odontoblasts or odontoblast-like cells, or an activity that promotes the mineralization.

The activity that promotes the differentiation of dental pulp cells into odontoblasts or odontoblast-like cells may be an activity that increases mRNA expression or protein expression of an odontogenic differentiation marker gene. The differentiation marker gene may be one or more selected from the group consisting of ALP, DMP-1 and DSPP.

The mineralization means that calcium and phosphorus, which are major components in dentin or enamel of teeth, are deposited as apatite to form teeth. The mineralization process plays an important role in the differentiation mechanism of odontoblasts in tooth development and stem cell-based tooth regeneration. Tooth mineralization is a phenomenon mainly caused by the deposition of tertiary dentin, which is generally characterized by discontinuity with secondary dentin due to highly irregular dentinal tubules. This characteristic acts to protect noxious materials from being transferred to the dental pulp tissue from the outside through the dentinal tubules. The mineralization may be measured by Alizarin S red (ARS) staining and its quantitative analysis, alkaline phosphatase (ALP) staining, activity, and RT-PCR (real time-PCR). However, the mineralization is not limited thereto and may be measured in a variety of ways. Thus, the activity that promotes the mineralization may be an activity that promotes the regeneration of teeth or dentin by increasing calcium or phosphorus deposition in teeth.

In one embodiment, as demonstrated by the experimental results of the specific examples of the present specification, it was confirmed that since the peptide of the present invention significantly increased the degree of ALP or ARS staining compared to the negative control group, the VnP-16 peptide induced the differentiation of dental pulp cells into odontoblasts or odontoblast-like cells and showed an activity to promote the mineralization (FIGS. 2A to 2D).

In one embodiment, the peptide of the present invention may promote the expression of one or more odontogenic differentiation marker genes selected from the group consisting of ALP, DMP-1 and DSPP in dental pulp cells, as demonstrated by the experimental results of the specific examples of the present specification.

In general, the composition of the present invention may further comprise a component having a dentin regeneration effect in addition to the above-described peptide as an active ingredient.

The composition of the present invention may comprise not only the peptide but also a polynucleotide encoding the peptide or a recombinant vector comprising the polynucleotide, as an active ingredient.

In the present invention, the term “polynucleotide” is a polymer of nucleotides in which nucleotide monomers are linked in a chain shape by covalent bonds, and refers to a DNA nucleic acid molecule or an RNA nucleic acid molecule of a certain length or longer, which is a polynucleotide encoding the peptide of the present invention.

In the polynucleotide of the present invention, various alterations may be made to the coding region within a range that does not change the amino acid sequence of the peptide expressed from the coding region in consideration of codons preferred in organisms to express the peptide, and various alterations or modifications may be made within a range that does not affect gene expression even in areas other than the coding region.

As long as the polynucleotide of the present invention encodes a peptide having an activity equivalent thereto, one or more nucleic acid bases may be mutated by substitution, deletion, insertion or a combination thereof, and these are also included in the scope of the present invention.

The recombinant vector of the present invention refers to an expression construct that is a means for stably expressing the peptide of the present invention by being introduced into a cell, and includes, for example, known expression vectors such as plasmid vectors, cosmid vectors, bacteriophage vectors and viral vectors, and such recombinant vectors may be easily prepared by those skilled in the art according to any known method using DNA recombinant technology.

Another aspect of the present invention provides a method of regenerating dentin, comprising the step of contacting a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide to dental pulp or dental pulp cells. Another aspect of the present invention provides a method of regenerating dentin, comprising the step of contacting a peptide comprising the amino acid sequence of SEQ ID NO: 1 (RVYFFKGKQYWE), a polynucleotide encoding the peptide or a recombinant vector comprising the polynucleotide to dental pulp or dental pulp cells.

On the one hand, the composition for regenerating dentin according to the present invention may also be provided in the form of a pharmaceutical composition to be described later.

### Pharmaceutical Composition for Preventing or Treating Dental Pulp Disease

Another aspect of the present invention provides a pharmaceutical composition for preventing or treating dental pulp disease, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

In the pharmaceutical composition, the peptide may be a peptide comprising the amino acid sequence of SEQ ID NO: 1.

The peptide of the present invention uses the contents described in the composition for regenerating dentin, and are not described redundantly. Since the peptide of the present invention has an activity to promote the regeneration of dentin in dental pulp cells, as described above, it may be used for preventing or treating dental pulp disease.

The dental pulp disease refers to a lesion occurring in the dental pulp tissue which is a soft connective tissue filling the pulp chamber inside the tooth and is richly distributed with nerves and blood vessels. When the dental pulp is exposed due to damage to dentin, and physical, chemical or bacterial stimuli are applied to the dental pulp, blood vessels in the dental pulp are initially dilated and congestion may be seen (pulp hyperemia), and if these stimuli continue, the dental pulp becomes inflamed (pulpitis). There is a difference in the degree of inflammation depending on the intensity of the stimulus and the presence or absence of bacterial infection, and if inflammation occurs due to the anatomical characteristics of the dental pulp piled up by hard dentin, circulation disorders are likely to occur, and if left alone, the dental pulp is likely to necrose. The causes of dental pulp disease are very diverse, but in most cases, it occurs by bacterial infection due to dental caries and infection into the dental pulp through perforations, fractures and cracks of the teeth and through periodontal pocket. It may also be caused by trauma, abrasion, cracked teeth, heat and friction from dental instruments during treatment, and the like. When the dental pulp disease worsens and inflammation spreads to the root and gingiva of the tooth, it may expand to periapical disease and periodontal disease. The dental pulp disease to be treated in the present invention may include, but is not limited to, for example, dentin hyperesthesia, pulp hyperemia, pulpitis, pulp degeneration, necrosis of pulp, or gangrene of pulp.

The term “preventing” of the present invention refers to all actions that inhibit or delay the onset of dental pulp disease by administering a pharmaceutical composition for preventing or treating the dental pulp disease, comprising the peptide of the present invention.

The term “treating” of the present invention refers to all actions that may be performed to treat dental pulp disease by administering a pharmaceutical composition comprising the peptide of the present invention as an active ingredient to a subject in need of treatment for the dental pulp disease to promote the regeneration of dentin or dental pulp tissue.

The pharmaceutical composition of the present invention comprises (i) a therapeutically effective amount of the above-described peptide of the present invention, a polynucleotide encoding the peptide, or a recombinant vector comprising the polynucleotide; and (ii) a pharmaceutically acceptable carrier.

The term “therapeutically effective amount” refers to an amount sufficient to achieve the therapeutic or preventive effect of the composition of the present invention on dental pulp disease.

The pharmaceutically acceptable carriers are those commonly used in formulations, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil.

The pharmaceutical composition of the present invention may further comprise, but is not limited to, a lubricant, a humectant, a sweetener, a flavouring agent, an emulsifier, a suspending agent, and a preservative, in addition to the above-mentioned ingredients. Suitable pharmaceutically acceptable carriers and agents are described in detail in Remington: The Science and Practice of Pharmacy, (21th ed., 2006, Williams & Wilkins).

The pharmaceutical composition of the present invention may be administered through all routes suitable for treating dental pulp disease, and for example, it may be administered orally or parenterally, wherein for parenteral administration, it may be applied directly to the area where the dental pulp disease occurs or performed by local administration, and besides, it may be administered intravenous injection, subcutaneous injection, intramuscular injection, intraperitoneal injection, transdermal administration, and the like.

The suitable dosage of the pharmaceutical composition of the present invention may vary depending on factors such as formulation method, the mode of administration, the age, body weight, sex and pathological condition of the patient, diet, the time of administration, the route of administration, the rate of excretion and response sensitivity, and usually, an effective dosage for desired treatment or prevention may be easily determined and prescribed by a skilled physician.

The daily dosage of the pharmaceutical composition of the present invention may be, but is not limited to, 0.00001-10,000 mg/kg, and various dosages may be administered depending on factors such as formulation method, the mode of administration, the age, body weight, sex and pathological condition of the patient, diet, the time of administration, the route of administration, the rate of excretion and response sensitivity.

The pharmaceutical composition of the present invention may be formulated in a single-dose form or packaged in multi-dose vessels using a pharmaceutically acceptable carrier and/or excipient, according to methods that may be easily carried out by those skilled in the art. In this case, the formulation may be in the form of a solution, suspension, or emulsion in oil or aqueous medium, or in the form of an extract, a powder, a granule, a tablet, or a capsule, and may further comprise a dispersing agent or a stabilizer.

The method of treating dental pulp disease using the pharmaceutical composition of the present invention may be, but is not limited to, direct pulp capping, and may be used in various methods of treating dental pulp disease, such as apexification, barrier technique, indirect pulp capping, pulpotomy, pulpectomy, induction of periapical hard tissue formation, guided bone regeneration (GBR), root perforation repair, and the like. Specifically, if the composition of the present invention is used in direct pulp-capping, which is a treatment method that helps healing and prevents secondary infection through application to the exposed nerve area, when dentin is damaged and the dental nerve of the dental pulp is exposed, it can effectively treat dental pulp disease.

The pharmaceutical composition of the present invention may further comprise a drug capable of controlling the regeneration and inflammatory response of dental pulp tissue or dentin. For example, the drug may include inhibitors of histone deacetylases, antioxidants, anti-inflammatory drugs whose main action is the inhibition of phospholipases and cyclooxygenases, regulators of Wnt signaling, and antibiotics. Specifically, the possible additional drug may be one or more drugs selected from the group consisting of trichostatin A, butyrate, valproic acid, apicidin; N-acetyl cysteine; aspirin, Tylenol (acetaminophen), ibuprofen, diclofenac, indomethacin, ketoprofen, Voltaren, feldene, Mobic; penicillin, cephalosporin, kanamycin, gentamicin, sisomicin, erythromycin, vancomycin, teicoplanin, quinolone, isoxazole derivatives, and lithium chloride.

According to another aspect of the present invention, there is provided a method of treating or preventing dental pulp disease, comprising the step of administering a therapeutically effective amount of a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide to a subject in need of treatment for the dental pulp disease. The subject may include a human or a non-human mammal.

### Composition for Root Canal Filling Material and Pulp-Capping Agent

Another aspect of the present invention provides a composition for a root canal filling material, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

Another aspect of the present invention provides a composition for a pulp-capping agent, comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide, as an active ingredient.

In the composition, the peptide may be a peptide comprising the amino acid sequence of SEQ ID NO: 1. In the present invention, the term “root canal capping” refers to the shaping of the root canal after cleaning and the permanent sealing of the root canal after drug treatment in dental caries treatment. The root canal filling material used for the root canal capping is divided into a gutta-percha that fills the root canal core and a root canal sealer that is responsible for sealing. Root canal filling material is a material that directly fills the empty space in order to seal the nerve and cell-rich dental pulp tissue in the dental crown area, by removing the damaged or infected dental pulp tissue and placing it on top of the cut dental pulp tissue in the process of dental caries treatment. In addition, the root canal sealer is used to seal the root canal after filling the empty space in the process of dental caries treatment, and acts to prevent microorganisms from entering the dental pulp or apical tissue or to trap and block microorganisms remaining in the root canal. In addition, it serves to prevent pain by closing the fine tooth root canal present in the root canal. The preferred root canal filling material should have biocompatibility, good sealing properties, and activity to promote the regeneration of damaged dental pulp tissue.

Since the peptide of the present invention promotes the behavior of dental pulp cells and the differentiation of odontoblasts to promote the regeneration of dentin, especially tertiary dentin, as described above, it may be included in a root canal filling material or pulp-capping agent to be used as an active ingredient.

The peptide of the present invention uses the contents described in the composition for regenerating dentin, and are not described redundantly.

The composition for a root canal filling material of the present invention may further comprise a material used as a dental root canal filling material.

The composition for a root canal filling material may include, for example, a zinc oxide-eugenol-based sealer, a resin-based sealer, a calcium hydroxide-based sealer, a glass ionomer-based sealer, a formaldehyde-containing sealer, gutta-percha, chloroperchar, a silicone-based sealer, a mineral trioxide aggregate (MTA)-based sealer, a calcium-silicate-phosphate-based bioceramic sealer, or a calcium phosphate sealer.

The zinc oxide-eugenol-based sealer may include zinc oxide and eugenol, or may include zinc oxide, a resin, and eugenol. The resin-based sealer may include an epoxy resin-based sealer or a methacrylate resin-based sealer. The calcium hydroxide-based sealer may include zinc oxide, eugenol and calcium hydroxide, or may include a resin and calcium hydroxide.

The composition for the root canal filling material may further comprise at least one of, for example, mineral trioxide aggregate (MTA), gutta percha, calcium oxide, zinc oxide, resin, epoxy resin, methacrylate resin, eugenol, aluminum oxide, aluminum silicate, silicon dioxide, silicone, salicylate, fatty acid, glass ionomer, calcium carbonate, calcium silicate, tricalcium silicate, calcium hydroxide, tricalcium phosphate, tetracalcium phosphate, octacalcium phosphate, hydroxyapatite, collagen, demineralized freeze-dried bone, indium foil, calcium sulfate, dentin or cementum chips, bone chips, titanium, chitosan, Teflon, and tantalum.

In addition, the composition for a root canal filling material may further comprise at least one contrast agent selected from barium oxide, barium sulfate, zirconium oxide, tantalum oxide, yttrium oxide, and calcium tungstate. In this case, the contrast agent may impart radiopacity to enable observation of the composition for a root canal filling material in a radiograph. However, the composition for a root canal filling material of the present invention is not limited to the above and may further comprise more various additives.

Another aspect of the present invention provides a method of capping a root canal, comprising the step of placing a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide in the root canal.

Another aspect of the present invention provides a method of capping a root canal, comprising the step of placing a peptide comprising the amino acid sequence of SEQ ID NO: 1 (RVYFFKGKQYWE), a polynucleotide encoding the peptide or a recombinant vector comprising the polynucleotide in the root canal.

The present invention provides a composition for a pulp-capping agent.

In the present invention, the pulp-capping agent refers to a material that is applied on top of the cut pulp tissue after removing the exposed dental pulp tissue in order to maintain the vitality of dental pulp-exposed teeth in partial pulpotomy, which is a conservative treatment of teeth.

The composition for a pulp-capping agent may further comprise the same material as the material added to the above-described composition for a root canal filling material, and the material to be further included in the composition for a pulp-capping agent uses the contents of the material to be added as described in the composition for a root canal filling material, and are not described redundantly to avoid excessive complexity of the specification.

Another aspect of the present invention provides a method of capping dental pulp, comprising the step of placing a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide or a recombinant vector comprising the polynucleotide in the dental pulp.

Another aspect of the present invention provides a method of capping dental pulp, comprising the step of placing a peptide comprising the amino acid sequence of SEQ ID NO: 1 (RVYFFKGKQYWE), a polynucleotide encoding the peptide or a recombinant vector comprising the polynucleotide in the dental pulp.

### Advantageous Effects

By using the vitronectin-derived functional peptide of the present invention, cell attachment, cell spreading and cell migration of dental pulp cells may be increased, and the differentiation of dental pulp cells into odontoblasts may be promoted. As such, since the peptide of the present invention has an activity that promotes the regeneration of dentin, it may be usefully used as an active ingredient in dental materials related to dentin regeneration, such as root canal filling materials and pulp-capping agents, and may also be used as a therapeutic agent for dental pulp disease.

However, the effects of the present invention are not limited to the effect mentioned above, and other effects that are not mentioned will be clearly understood by those skilled in the art from the following description.

### DESCRIPTION OF DRAWINGS

FIG. 1A shows human dental pulp cells (hDPCs) attached to peptide-treated plates in serum-free medium for 1 hour (upper panel) and spreading of human dental pulp cells after 3 hours (lower panel).

FIG. 1B shows the quantification of the degree of cell attachment and spreading of human dental pulp cells with respect to immobilized peptides.

FIG. 1C shows scratch wound migration after applying scratch stimuli to human dental pulp cells reaching 80% confluence to induce horizontal migration.

FIG. 1D shows the result of measuring the migration area of the cells at 0, 24 and 48 hours after applying scratch stimuli to the human dental pulp cells.

FIG. 1E is the result showing the viability of human pulp cells at 24 and 48 hours after treatment with the VnP-16 peptide.

FIGS. 2A to 2E show experimental results showing the activity of the VnP-16 peptide to promote the differentiation and mineralization of human pulp cells into odontoblast-like cells.

FIG. 2A shows the results of performing alkaline phosphatase (ALP) staining on human dental pulp cells after induction of odontogenic differentiation for 7 days by treatment with the VnP-16 peptide.

FIG. 2B shows the results of performing Alizarin Red S (ARS) staining on human dental pulp cells after induction of odontogenic differentiation for 21 days by treatment with the VnP-16 peptide.

FIG. 2C shows the quantification of alkaline phosphatase (ALP) staining results of odontogenic differentiation-induced human odontogenic cells.

FIG. 2D shows the quantification of Alizarin Red S (ARS) staining results of odontogenic differentiation-induced human odontogenic cells.

FIG. 2E shows the results of performing quantitative real-time polymerase chain reaction (qRT-PCR) on ALP, DMP1 and DSPP genes in cells harvested on days 3, 7 and 14 after induction of odontogenic differentiation.

FIG. 3A shows the result of histologically and quantitatively analyzing the inflammatory response in the exposed dental pulp area 2 weeks after the pulp exposure surgery of a rat.

FIG. 3B shows the result of histologically and quantitatively analyzing the degree of reparative dentin formation in the exposed dental pulp area 2 weeks after the pulp exposure surgery of a rat.

FIG. 3C shows a cross section after H&E staining of the exposed dental pulp area 2 weeks after the pulp exposure surgery of a rat, wherein a slight inflammatory response could be confirmed in all groups, and it could be confirmed that no hard tissue was formed in all groups except for the MTA group and the VnP-16 group.

FIG. 4A the result of histologically and quantitatively analyzing the inflammatory response in the exposed dental pulp area 4 weeks after the pulp exposure surgery of a rat.

FIG. 4B shows the result of histologically and quantitatively analyzing the degree of reparative dentin formation in the exposed dental pulp area 4 weeks after the pulp exposure surgery of a rat.

FIG. 4C shows a cross section after H&E staining of the exposed dental pulp area 2 weeks after the pulp exposure surgery of a rat, wherein hard tissues were formed in all groups, but in particular, reparative dentin was formed thicker and more homogeneously in the VnP-16 group than in the MTA group or the rhBMP-2 group, which are positive controls.

FIG. 5 shows results of performing the immunohistochemical staining using an anti-DSP antibody 2 weeks after the pulp exposure surgery of a rat, wherein DSP was detected scattered along the cells in all groups except for the resin-only group. The MTA group and the VnP-16 group showed a high intensity inside the mineralized tissue.

FIG. 6 shows the results of performing immunohistochemical staining using an anti-DSP antibody 4 weeks after the pulp exposure surgery of a rat. At 4 weeks, the resin-only group showed no staining. As hard tissue formation progressed, the degree of staining decreased in the center of the exposed pulp, and rather, it was observed that the cells surrounding the hard tissue were stained in most groups.

### BEST MODE

Hereinafter, the present application will be described in detail by examples. However, the following examples are only for specifically illustrating the present application, and the content of the present application is not limited by the following examples.

### Example 1

### Preparation of Peptides and Reagents

All peptides of the present invention were synthesized on a Pioneer Peptide Synthesizer (Applied Biosystems) using the 9-fluorenylmethoxycarbonyl (Fmoc)-based method, with a C-terminal amide. The peptides were purified and characterized by Peptron company (Daejeon, Korea). All peptides used in the present invention had a purity of 97% or more. In the experiment, the VnP-16 peptide of the present invention (RVYFFKGKQYWE; SEQ ID NO: 1) was used as an experimental group, human plasma vitronectin purchased from Millipore (Bedford, MA, USA) was used as a positive control, and scrambled peptide (SP) (FVWRQFYKYEKG; SEQ ID NO: 3) was used as a negative control.

### Example 2

### Cell Culture

Human dental pulp cells (hDPCs) were collected from the patient's third molars after patient consent and approval from the Institutional Review Board (IRB) of Seoul National University Dental Hospital (IRB No. CRI19007) were obtained. The hDPCs were passaged three times in Dulbecco's modified Eagle's medium (DMEM; Gibco BRL, Carlsbad, CA, USA) supplemented with 10% fetal bovine serum (FBS) and 1% penicillin/streptomycin (10,000 U/ml penicillin, 10,000 μg/ml streptomycin; Gibco, Paisley, Scotland). In order to induce odontogenic differentiation, the hDPCs were then cultured using DMEM supplemented with 10% FBS, 1% penicillin/streptomycin, 10 mM β-glycerophosphate, 200 μM ascorbic acid, and 0.1 μM dexamethasone.

### Example 3

### Cell Viability Assay

The viability of hDPCs in the presence of VnP-16 was investigated using an Cytox Cell Viability Assay Kit (DoGenBio, Seoul, Korea). A 96-well culture plate was treated with 50, 100 and 200 μg/ml VnP-16, and then 1.5×10⁴ cells were plated into each well, and incubated for 24 or 48 hours at 37° C. The water-soluble tetrazolium salt reagent (10 [symbol] ) was added to each well, and the plate was incubated at 37ºC for 2 hours. Finally, absorbance at 450 nm was measured using a microplate reader (Epoch2, Bio-Tek, VT, USA).

### Example 4

### hDPCs Cell Attachment and Spreading Assays

First, for the cell attachment assay, 48-well culture plates were coated with human plasma vitronectin (0.26 μg/cm2) at 4° C. for 24 hours. The SP and VnP-16 (10.5 μg/cm2) were also coated on the plates by drying at room temperature for 24 hours. The cell attachment assay was determined from a dose-response curve, and the lowest concentration that showed the maximal attachment was used. The peptide-coated plates were blocked with 1% heat-inactivated bovine serum albumin (BSA) in phosphate-buffered saline (PBS) at 37° ° C. for 1 hour, and then washed with PBS. The hDPCs (5×10⁴ cells/250 μL) were added to each plate and incubated at 37° ° C. for 1 hour, and then unattached cells were removed by rinsing the plates twice with PBS. The attached cells were fixed with 10% formalin for 15 minutes and stained with 0.5% crystal violet for 1 hour. The plates were then gently washed three times with double-distilled water. Afterwards, the contents of each well were solubilized in 2% sodium dodecyl sulfate for 5 minutes, and then absorbance at 570 nm was measured using a microplate reader.

In addition, for the cell spreading assay, hDPCs (3×10⁴ cells/250 μL) were incubated at 37° ° C. for 3 hours on each peptide-coated plate in the same manner as above. The attached cells were fixed with 10% formalin and stained with 0.5% crystal violet for 1 hour. Then, the plates were gently washed three times with PBS, and then the degree of cell spreading was assessed by measuring the surface area of the cells using Image Pro Plus software (version 4.5; Media Cybernetics, Silver Spring, MD, USA).

### Example 5

### Scratch Wound Migration Assay

In order to evaluate the migration ability and wound healing ability of hDPCs, the scratch assay was used. The hDPCs were plated in a 24-well plate and incubated in complete medium until 80% confluence. Then, the cells were passaged in serum-free medium for 24 hours, and then stimulated by scratching with a 200 μl pipette tip. After PBS washing to remove cell debris, the cultures were treated with vehicle (10% DMSO), vitronectin (1.25 μg/ml), SP (50 μg/ml), or VnP-16 (50 μg/ml). Pictures were taken using a microscope (Nikon, Tokyo, Japan) at 0, 24 and 48 hours after treatment. The data were analyzed using Image Pro Plus software (version 4.5; Media Cybernetics, MD, USA) to compare the percentage of scratch closure from 0 hour to 48 hours time point. The percentage of: scratch closure was calculated by the following equation: Scratch closure %=[A (0)−A(t)/A(0)]×100. Wherein the scratch area at time zero is “A (0)”; and the area after the incubation time (t) is “A(t)”. The scratch wound migration assay was performed in triplicate.

### Example 6

### Alkaline Phosphatase (ALP) and Alizarin Red S (ARS) Staining

ALP staining was performed 7 days after odontogenic induction using a tartrate-resistant acid phosphatase (TRAP)/ALP double-stain kit (Takara, Shiga, Japan) according to the manufacturer's protocol. ARS staining was performed 3 weeks after induction. For ARS staining, cells were fixed with ice-cold 95% ethanol at −20° C. for 30 minutes, and then stained with 40 mM Alizarin red S solution (pH 4.2) for 1 hour. The stained cells were washed five times with double-distilled water, and then rinsed with PBS for 15 minutes. In order to visualize mineral deposits, the plates were photographed using a microscope (Nikon, Tokyo, Japan).

### Example 7

### Quantitative Real-Time Polymerase Chain Reaction (gRT-PCR)

Isolation of total RNA from human dental pulp cells (hDPCs) was performed using RNeasy® MiniKit (QIAGEN, Valencia, CA, USA) according to the manufacturer's instructions. CDNA was prepared using SuperScript® IV reverse transcriptase (Invitrogen, CA, USA), and PCR amplification of specific marker genes was performed. The CDNA was amplified using SYBR® Premix Ex Taq™ (Takara), with each primer in [Table 1] below at a concentration of 200 nM. The cycle threshold (CT) values were determined by automated threshold analysis using Sequenced Detection Software (version 1.3; Applied Biosystems), and further analyzed using Microsoft Excel. The relative expression level of each target mRNA was analyzed using the comparative cycle threshold method according to the manufacturer's protocol (Applied Biosystems).

TABLE 1

| SEQ | | | |
|---|---|---|---|
| ID NO: | Gene | Primer | Base Sequence (5′→3′) |
| 4 | GAPDH | Forward | GTATCCGTTGTGGATCTGAC |
| 5 | | Reverse | TGTTGAAGTCACAGGAGACA |
| 6 | ALP | Forward | ACTGGTACTCGGACAATGAG |
| 7 | | Reverse | AGACATAGTGGGAGTGCTTG |
| 8 | DMP-1 | Forward | CACACAGTCCAGTGAAGACA |
| 9 | | Reverse | ACCCGATATTCCTCACTCTC |
| 10 | DSPP | Forward | CAGAGGTCAGGGTTCAGTTA |
| 11 | | Reverse | GAGATTCTGGTTGTCCTGTG |
| 12 | OC | Forward | CTCTCTCTGCTCACTCTGCT |
| 13 | | Reverse | CGGAGTCTATTCACCACCT |
| 14 | RUNX2 | Forward | TACTTCGTCAGCGTCCTATC |
| 15 | | Reverse | CCAGACAGACTCATCCATTC |

[In Table 1, GAPDH: glyceraldehyde-3-phosphate dehydrogenase; ALP: alkaline phosphatase; DMP-1: dentin matrix protein-1; DSPP: dentin sialophosphoprotein; OC: osteocalcin; RUNX2: runt-related transcription factor 2]

### Example 8

### Rat Tooth Pulp Exposure

Seven-week-old male Sprague-Dawley rats were purchased from the OrientBio after approval from the Institutional Animal Care and Use Committee of Seoul National University (No. SNU-180109-2) was obtained. The rats were used after 1-week acclimation under standard animal housing conditions (12-h light and 12-h dark cycles with free access to food and water) and randomly divided into the following six groups for the pulp-capping procedure: self-adhesive resin cement (Resin) group, ProRoot MTA (MTA) group, vehicle (Veh) group, rhBMP-2 group, SP group, and VnP-16 group. The Resin group and Veh group were used as negative controls and the MTA group and rhBMP-2 group were used as positive controls. Resin cement (Rely X U-200, 3M ESPE, MN, USA) was used as the final restoration in all experimental groups.

Specifically, the rats were anesthetized by the intraperitoneal injection of 31.25 mg/kg tiletamine/zolazepam and 8.75 mg/kg xylazine. After ascertaining the general anesthesia, the oral cavity was opened with a customized wire to expose the lower first molars. After cleaning and sanitizing with both 3% H₂O₂ and a 10% Betadine-soaked cotton, the center of the occlusal surface was carefully punctured using a #¼ round bur (0.5 mm in diameter) with a high-speed hand piece. MTA (ProRoot MTA, Dentsply Sirona, York, PA, USA) was mixed according to the manufacturer's instruction and the mixture was applied to the exposed pulp. Absorbable collagen sponges (BiolandKorea, Cheonan, Korea) were loaded with vehicle (10 μL of DMSO and 5 μL of DDW) (vehicle (Vhe) group), rhBMP-2 (2 μg per scaffold, 15 μL volume; Pepro-Tech, Rocky Hill, NJ, USA) (rhBMP-2 group), or synthetic peptides SP or VnP-16 (2 mg per scaffold, 15 μL volume) (SP and VnP-16 groups), respectively, and then applied to the exposed pulp. The punctured sites were covered with resin cement after the application of MTA or the collagen sponge. In the Resin group, resin cement was directly applied to the punctured site. The sharp cusp tips of the upper molar and lower first molar were grinded using a football shaped diamond bur in order to prevent tooth fractures by mastication.

### Example 9

### Histologic Analyses

Rats were histologically analyzed 2 and 4 weeks after pulp exposure surgery. Whole mandibles were fixed f in 4% paraformaldehyde at 4° ° C. for 24 hours. For histological staining, the specimens were decalcified with 12% EDTA for 4 weeks and embedded in paraffin. The paraffin-embedded samples were sectioned to a thickness of 5 μm, and then these sections were treated with anti-DSP (MABT37; Millipore, Billerica, MA, USA) according to the manufacturer's protocol, and the samples were counterstained with a hematoxylin and eosin (H&E) solution and observed under a microscope. Each sample was evaluated and scored for inflammatory response and hard tissue formation according to the criteria presented in [Table 2] below. In this study, reparative dentin formed under the pulp-exposed site, which is distinguished from dentin regenerated under the existing dentin, was evaluated.

TABLE 2

| Score | Inflammatory Cell Infiltration | Hard Tissue Formation |
|---|---|---|
| 1 | None: No or very few | Heavy: The dental pulp area |
| | inflammatory cells | with exposed continuous dentin |
| | | bridges is completely restored. |
| 2 | Mild: Inflammatory cells only | Moderate: Discontinuous dentin |
| | next to the exposure of the | bridges are formed and more |
| | dentin bridges or pulp-exposed | than half of the exposed pulp |
| | areas/reached up to ⅓ of the | area is restored. But not |
| | root canal pulp tissue | completely restored. |
| 3 | Moderate: Inflammatory cells | Slight: Less than 50% of diffuse |
| | are observed from the crown | dentin bridges are formed in the |
| | pulp tissue to two-thirds of | exposed pulp area. |
| | the root canal pulp tissue. | |
| 4 | Severe: More than two-thirds | None: No hard tissue formation |
| | of all crown pulp/root canal | |
| | pulp tissues are infiltrated | |
| | or necrotized. | |

### Example 10

### Statistical Analysis

All data tested in the examples of the present invention are presented as the mean±standard deviation (SD), and statistical analysis of the results was performed using the STATISTICA 6.0 software package (StatSoft). Results were compared using an analysis of variance test. When significant differences were found, pairwise comparisons were performed using Scheffe's adjustment. Statistical significance was calculated using the two-tailed student's t-test. A difference in P-values of <0.05 were considered statistically significant.

### Example 11

### VnP-16 Promotes the Attachment, Spreading and Wound Migration of hDPCs In Vitro

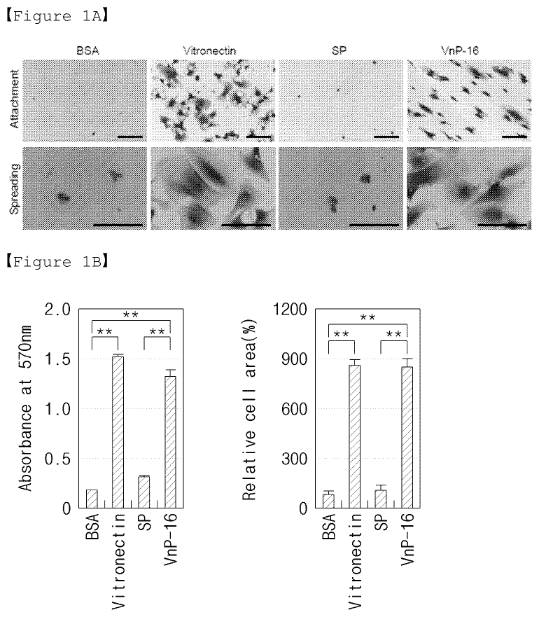

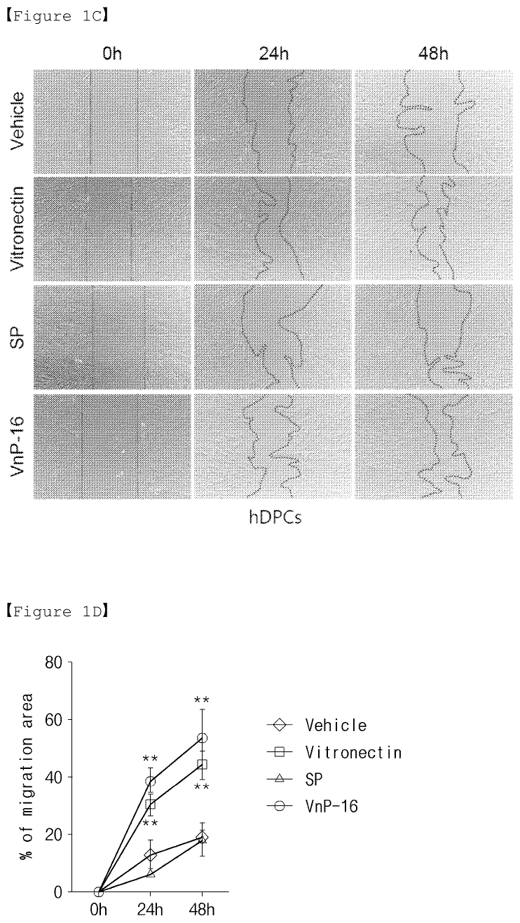

In order to investigate whether VnP-16 could induce hDPC behavior, cell attachment, cell spreading and scratch wound migration assays of hDPCs were performed according to the above-described examples. As a result, human plasma vitronectin strongly promoted cell attachment and spreading. VnP-16 also induced cell attachment and spreading at higher levels than the BSA control or SP control, which were confirmed at levels comparable to those of vitronectin (FIGS. 1A and 1B). As a result of scratch analysis, it was also confirmed that vitronectin and VnP-16 strongly promoted the wound migration of hDPCs (FIGS. 1C and 1D). Moreover, it was confirmed that VnP-16 had no cytotoxicity to hDPCs, and thus had cytocompatibility (FIG. 1E). These experimental results demonstrate that VnP-16 is a peptide with an activity that plays an important role in promoting the cell attachment and spreading of hDPCs.

### Example 12

### VnP-16 Promotes the Differentiation of hDPCs into Odontoblast-Like Cells and the Mineralization

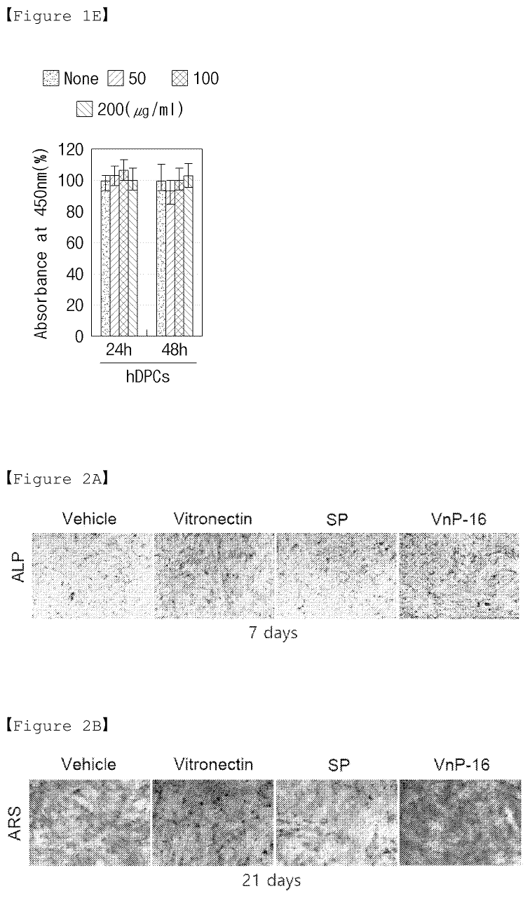

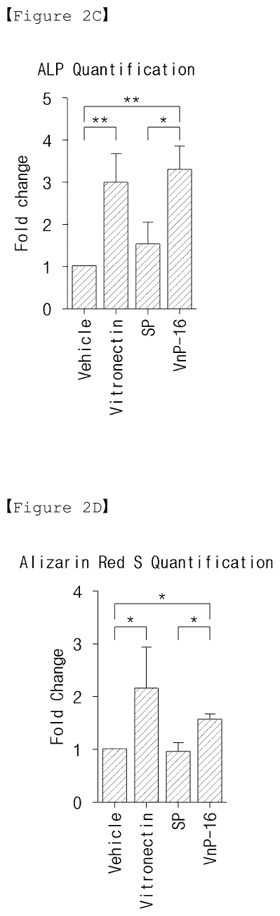

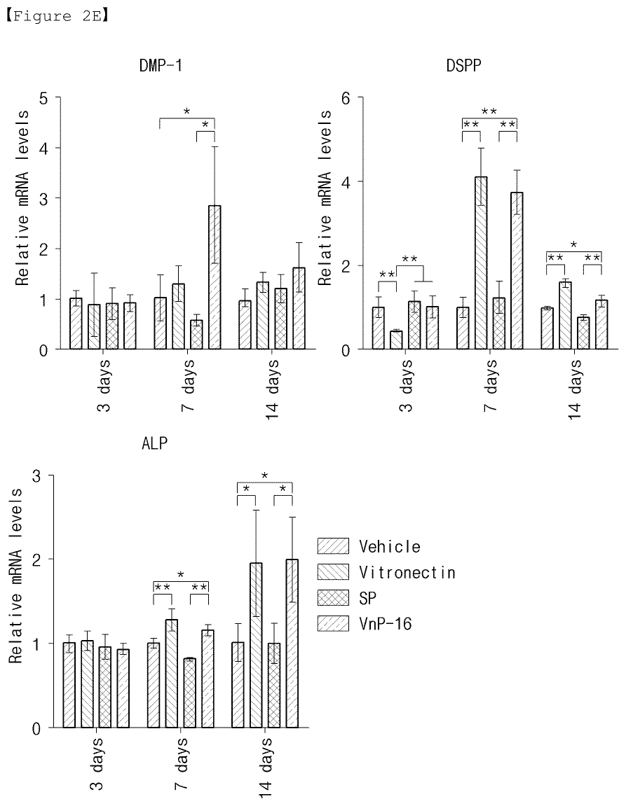

In order to assess the effects of VnP-16 on odontoblastic differentiation, hDPCs were cultured in Osteoblast Differentiation Medium (Cell Applications, San Diego, CA, USA) for 7 days (ALP) or 21 days (ARS). It could be confirmed that the VnP-16 showed odontogenic differentiation and mineralization by increased ALP staining and ARS staining at levels similar to those of vitronectin (FIGS. 2A to 2D). As a result of qRT-PCR, it was also confirmed that the VnP-16 upregulated odontogenic markers including ALP, DMP-1 and DSPP (FIG. 2E). These experimental results indicate that the VnP-16 promotes the differentiation and mineralization of hDPCs to an extent comparable to that achieved by vitronectin.

### Example 13

### VnP-16 Promotes Reparative Dentin Formation In Vivo

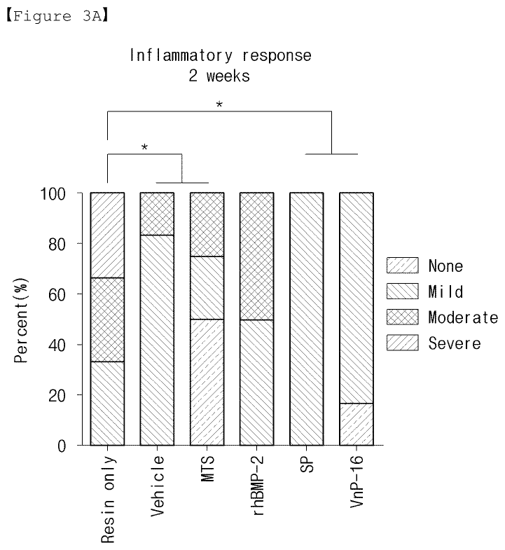

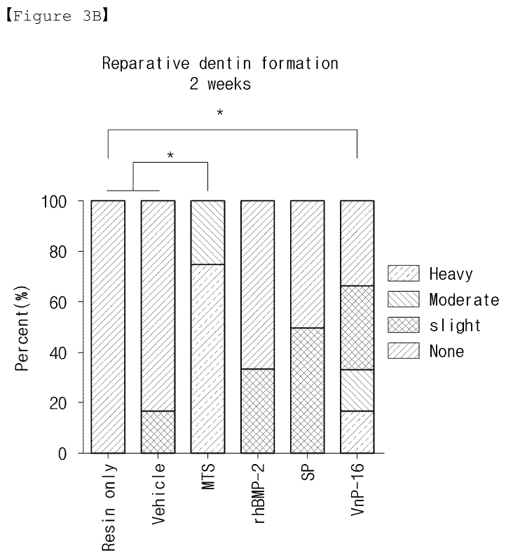

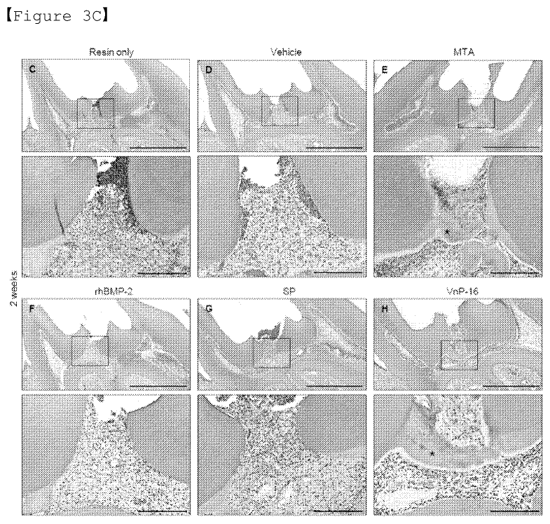

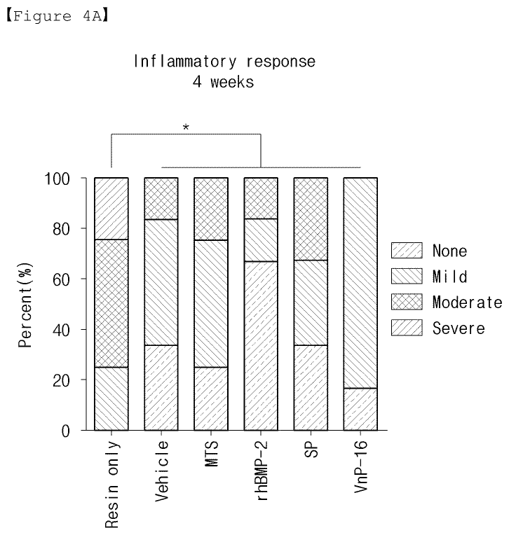

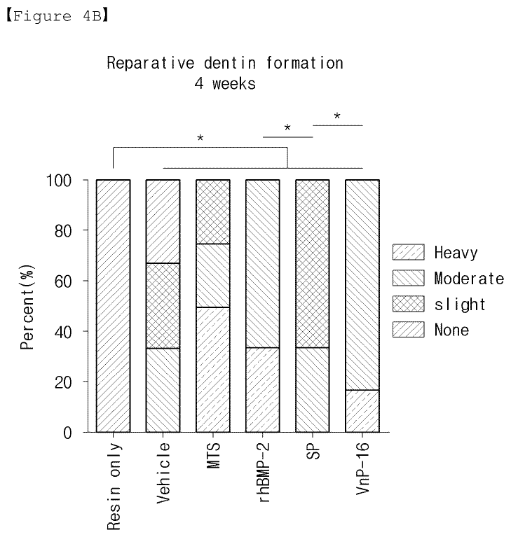

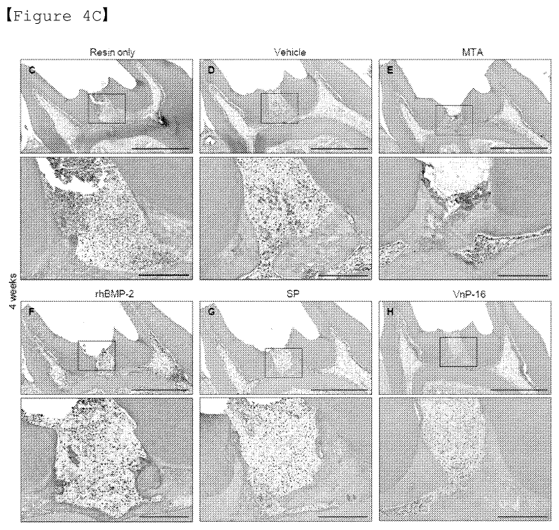

The in vivo potential of VnP-16 to induce reparative dentin formation was investigated in a rat pulp exposure model, and the inflammatory pulpal responses were evaluated (FIGS. 3 and 4).

At 2 weeks after pulp exposure surgery, no specimens showed a severe inflammatory pulpal response except for the resin-only group. A moderate inflammatory response was seen in 50% (3 out of 6 specimens) of the rhBMP-2 group, and in one specimen from the resin-only group (3 specimens), vehicle group (6 specimens) and MTA group (4 specimens) (FIG. 3A). Newly formed mineralized tissues were hardly detected at the pulp-exposed sites in the resin-only group, vehicle group, rhBMP-2 group and SP group (FIG. 3B), whereas reparative dentin completely covering the pulp-exposed site was formed in 3 out of 4 specimens among the MTA group and 1 out of 6 specimens among the VnP-16 group (FIG. 3C, bottom panels). In the MTA and VnP-16 groups, it could be seen that the cells lining the pulp showed a structure with a nucleus skewed toward the pulp, and were aligned at the boundary between the formed hard tissue bridges and the pulp, and thus they are similar to odontoblasts.

At 4 weeks after pulp exposure surgery, 75% (3 out of 4) of the resin-only group showed a moderate to severe inflammatory response, and the other groups showed mild to no inflammatory responses from 67% to 100% (FIG. 4A). All groups showed the tendency of reparative dentin formation with a slight to heavy score, but no dentin formation was detected in one specimen among the resin-only group and one specimen among the vehicle group. All specimens among the rhBMP-2 and VnP-16 groups showed moderate and heavy scored reparative dentin formation (FIG. 4B). The MTA, rhBMP-2, SP and VnP-16 groups also showed similar levels of reparative dentin formation at 4 weeks, but the quality of the reparative dentin was different among groups (FIG. 4C). All specimens among the SP group showed hard tissue formation, but included a non-tubular and large cell-inclusion structure. It was observed that the VnP-16 group showed thicker and more homogeneous reparative dentin formation compared to that in the MTA and rhBMP-2 groups, which were positive controls.

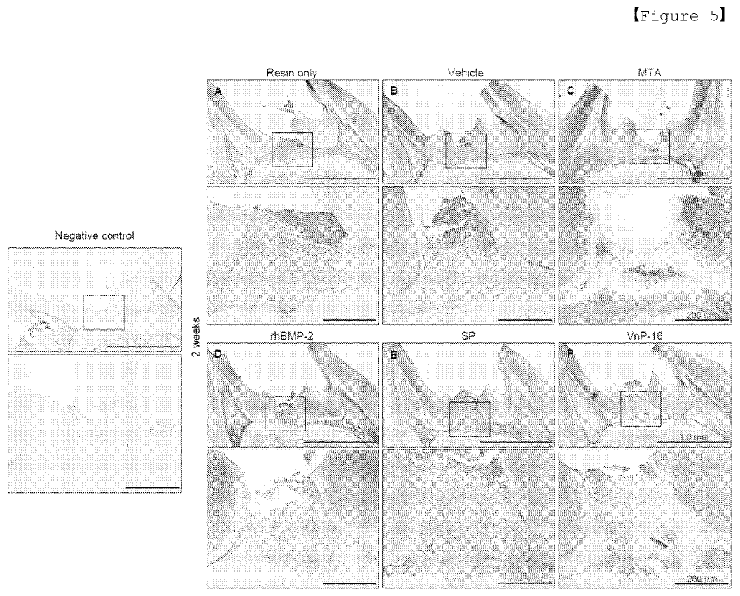

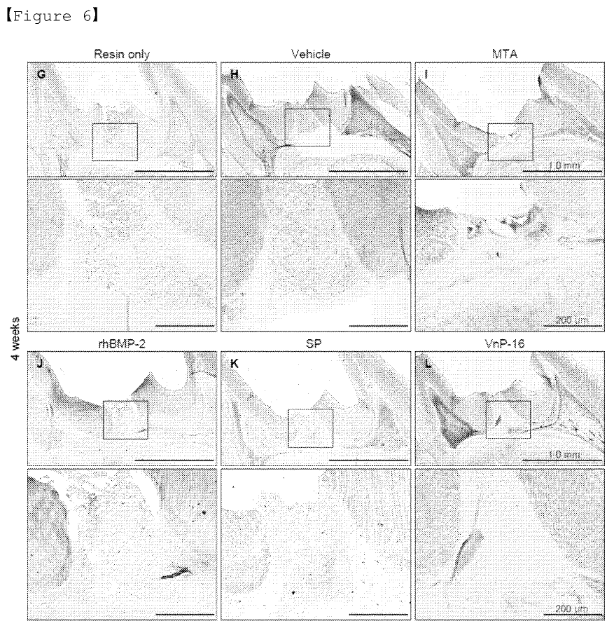

As shown in FIGS. 5 and 6, it could be confirmed that the immunohistochemistry staining for DSP showed results consistent with the H&E staining results, showing notable staining patterns in the dentin and around newly formed hard tissues in all groups. These experimental results indicate that the VnP-16 peptide has an activity to promote reparative dentin formation without an inflammatory response, which is superior to vitronectin and comparable to MTA and rhBMP2.

1.-16. (canceled)

17. A method of regenerating dentin, comprising contacting dental pulp or dental pulp cells with a composition comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide, or a recombinant vector comprising the polynucleotide.

18. The method of claim 17, wherein the composition comprises a peptide comprising the amino acid sequence of SEQ ID NO: 1, a polynucleotide encoding the peptide, or a recombinant vector comprising the polynucleotide.

19. The method of claim 17, wherein the dentin is reparative dentin or reactionary dentin.

20. The method of claim 17, wherein the dentin is dental pulp-exposed dentin.

21. The method of claim 17, wherein the peptide promotes the differentiation of dental pulp cells into odontoblasts or odontoblast-like cells.

22. The method of claim 17, wherein the peptide promotes the behavior of dental pulp cells.

23. The method of claim 22, wherein the behavior of the dental pulp cells is attachment, spreading or migration of the dental pulp cells.

24. The method of claim 17, wherein the peptide promotes the mineralization of dental pulp cells or dentin.

25. The method of claim 17, wherein the peptide increases the expression of one or more genes selected from the group consisting of ALP, DMP-1 and DSPP.

26. A method of treating dental pulp disease, comprising administering to a subject in need of treatment for the dental pulp disease a pharmaceutical composition comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide, or a recombinant vector comprising the polynucleotide.

27. The method of claim 26, wherein the peptide is a peptide comprising the amino acid sequence of SEQ ID NO: 1.

28. The method of claim 26, wherein the dental pulp disease is dentin hyperesthesia, pulp hyperemia, pulpitis, pulp degeneration, necrosis of pulp, or gangrene of pulp.

29. A method of capping a root canal, comprising placing a composition comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide, or a recombinant vector comprising the polynucleotide in the root canal.

30. The method of claim 29, wherein the peptide is a peptide comprising the amino acid sequence of SEQ ID NO: 1.

31. A method of capping dental pulp, comprising placing a composition comprising a peptide consisting of 12 to 173 contiguous amino acid sequences comprising the amino acid sequence of SEQ ID NO: 1 (RVYFFKGKQYWE) within the amino acid sequence of SEQ ID NO: 2, a polynucleotide encoding the peptide, or a recombinant vector comprising the polynucleotide in the dental pulp.

32. The method of claim 31, wherein the peptide is a peptide comprising the amino acid sequence of SEQ ID NO: 1.


SEQUENCE LISTING (15 sequences)

SEQ ID NO: 1 - protein, 12 aa, synthetic construct
RVYFFKGKQYWE

SEQ ID NO: 2 - protein, 173 aa, synthetic construct
PAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFW

SEQ ID NO: 3 - protein, 12 aa, synthetic construct
FVWRQFYKYEKG

SEQ ID NO: 4 - DNA, 20 bp, synthetic construct
  source 1..20: mol_type = other DNA
gtatccgttgtggatctgac

SEQ ID NO: 5 - DNA, 20 bp, synthetic construct
  source 1..20: mol_type = other DNA
tgttgaagtcacaggagaca

SEQ ID NO: 6 - DNA, 20 bp, synthetic construct
  source 1..20: mol_type = other DNA
actggtactcggacaatgag

SEQ ID NO: 7 - DNA, 20 bp, synthetic construct
  source 1..20: mol_type = other DNA
agacatagtgggagtgcttg

SEQ ID NO: 8 - DNA, 20 bp, synthetic construct
  source 1..20: mol_type = other DNA
cacacagtccagtgaagaca

SEQ ID NO: 9 - DNA, 20 bp, synthetic construct
  source 1..20: mol_type = other DNA
acccgatattcctcactctc

SEQ ID NO: 10 - DNA, 20 bp, synthetic construct
  source 1..20: mol_type = other DNA
cagaggtcagggttcagtta

SEQ ID NO: 11 - DNA, 20 bp, synthetic construct
  source 1..20: mol_type = other DNA
gagattctggttgtcctgtg

SEQ ID NO: 12 - DNA, 20 bp, synthetic construct
  source 1..20: mol_type = other DNA
ctctctctgctcactctgct

SEQ ID NO: 13 - DNA, 19 bp, synthetic construct
  source 1..19: mol_type = other DNA
cggagtctattcaccacct

SEQ ID NO: 14 - DNA, 20 bp, synthetic construct
  source 1..20: mol_type = other DNA
tacttcgtcagcgtcctatc

SEQ ID NO: 15 - DNA, 20 bp, synthetic construct
  source 1..20: mol_type = other DNA
ccagacagactcatccattc